(19) United States
(12) Patent Application Publication
(10) Pub. No.: US 2018/0308587 A1
(43) Pub. Date: Oct. 25, 2018

(54) COMMUNICATION HUB FOR PATIENT PHYSIOLOGICAL PARAMETERS

(71) Applicant: Draeger Medical Systems, Inc.

(72) Inventors: Rajesh Rane, Cambridge, MA (US); Michael D. Hirst, Webster, MA (US); William T. Buresh, II, Salem, NH (US)

(21) Appl. No.: 15/523,858

(22) PCT Filed: Nov. 25, 2014
(86) PCT No.: PCT/US2014/067508

§ 371 Date: May 2, 2017

(51) Int. Cl. G16H 80/00; A61B 5/00; H04L 12/44; G16H 10/60; H04L 29/08

(52) U.S. Cl. CPC G16H 80/00; A61B 5/0004; A61B 5/002; H04L 2012/445; G16H 10/60; H04L 67/12; H04L 12/44

(57) ABSTRACT

A patient parameter communication hub includes a plurality of patient-parameter-connectors, a data conversion unit, a medical-analysis station connector, and a data-network connector. The plurality of patient-parameter-connectors are adapted for receiving physiological parameter data in a first format. The physiological parameter data are generated by one or more physiological parameter data generators. The data conversion unit is adapted for converting the received physiological parameter data from the first format to a second format. The medical-analysis station connector is adapted for transmitting the received physiological parameter data in the second format to a medical-analysis station for processing and for receiving processed physiological parameter data in the second format from the medical-analysis station. The data-network connector adapted for transmitting the received processed physiological parameter data in the second format over a data-network to one or more destinations. Related apparatus, systems, techniques, and articles are also described.

### TECHNICAL FIELD

The subject matter described herein relates to multi-connection hubs providing communication capabilities between patient parameter sources, patient monitors, and data networks.

### BACKGROUND

Hospitals can monitor physiological parameters of patients from when the hospital first admits a patient until they release the patient. One or more patient monitoring devices, such as a heart rate monitor, an ECG monitor, blood oxygen (SpO2) monitor, and so forth, can perform the monitoring. These monitoring devices may be separate pieces of equipment and can be manufactured by different manufacturers. The monitoring equipment can include connections to the patient necessary to measure the physiological parameter and a display device of the type necessary to display the physiological parameter in an appropriate manner. A healthcare worker, such as a nurse, can visit a patient's location and look at each separate system to accumulate the patient's vital signs.

### SUMMARY

In an aspect, a patient parameter communication hub includes a plurality of patient-parameter-connectors, a data conversion unit, a medical-analysis station connector, and a data-network connector. The plurality of patient-parameter-connectors are adapted for receiving physiological parameter data in a first format. The physiological parameter data are generated by one or more physiological parameter data generators. The data conversion unit is adapted for converting the received physiological parameter data from the first format to a second format. The medical-analysis station connector is adapted for transmitting the received physiological parameter data in the second format to a medical-analysis station for processing and for receiving processed physiological parameter data in the second format from the medical-analysis station. The data-network connector adapted for transmitting the received processed physiological parameter data in the second format over a data-network to one or more destinations.

In another aspect, data in a first format and characterizing patient physiological parameters measured by one or more physiological parameter data generators can be received by one of a plurality of patient-parameter-connectors. The received patient physiological parameter data can be converted by a data conversion unit from the first format to a second format according to an Ethernet protocol. The patient physiological parameter data can be transmitted by at least an Ethernet switch having three or more connectors. The patient physiological parameter data can be transmitted in the second format on a first Ethernet connector to a medical-analysis station.

In yet another aspect, a system includes a plurality of serial ports, an Ethernet switch, and a data conversion unit. The Ethernet switch includes a medical-analysis station port and a data-network port. The data conversion unit is coupled to the plurality of serial ports and the Ethernet switch. The data conversion unit includes at least one processor and executable instructions stored on memory, which, when executed by the at least one processor, causes the at least one processor to receive, on one of the plurality of patient-parameter-connectors, data in a first format and characterizing patient physiological parameters measured by one or more physiological parameter data generators. The received patient physiological parameter data is converted from the first format to a second format according to an Ethernet protocol. The patient physiological parameter data is transmitted by the medical-analysis station port and in the second format to a medical-analysis station.

In yet another aspect, a system includes a medical-analysis station, one or more physiological parameter data generators, and a patient parameter communication hub. The patient parameter communication hub includes one or more serial ports operatively in communication with the one or more physiological parameter data generators, an Ethernet switch, and a data conversion unit. The Ethernet switch includes a medical-analysis station port and a data-network port. The medical-analysis station port is operatively in communication with the medical-analysis station. The data conversion unit is coupled to the one or more serial ports and the Ethernet switch. The data conversion unit includes at least one processor and executable instructions stored on memory, which, when executed by the at least one processor, causes the at least one processor to receive, on one of the plurality of patient-parameter-connectors, data in a first format and characterizing patient physiological parameters measured by the one or more physiological parameter data generators. The received patient physiological parameter data is converted from the first format to a second format according to an Ethernet protocol. The patient physiological parameter data is transmitted by the medical-analysis station port in the second format to the medical-analysis station.

In yet another aspect, a patient parameter communication hub includes a plurality of serial ports for receiving physiological parameter data in a serial format, a data conversion unit, and an Ethernet switch. The physiological parameter data is generated by a plurality of physiological parameter data generators. The data conversion unit includes at least one processor and executable instructions stored on memory, which, when executed by the at least one processor, causes the at least one processor to convert the physiological parameter data received in the serial format to a data packet format according to an Ethernet protocol for transmission on a first Ethernet port. The Ethernet switch includes a medical-analysis station port and a data-network port. The medical-analysis station port is for transmitting the physiological parameter data in the data packet format to a medical-analysis station for processing and for receiving processed physiological parameter data in the data packet format from the medical-analysis station. The data-network port is for transmitting the received processed physiological parameter data in the data packet format over a data-network to one or more destinations.

One or more of the following features can be included in any feasible combination. For example, the first format can include a serial format. The second format can include an Ethernet format. The plurality of patient-parameter-connectors can include a serial port. The medical-analysis station connector can include a first Ethernet port. The data-network connector can include a second Ethernet port.

The data conversion unit can include at least one processor and executable instructions stored on memory, which, when executed by the at least one processor, causes the at least one processor to convert the physiological parameter data received on one or more of the plurality of patient-parameter-connectors to data packets according to an Ethernet protocol for transmission on the first Ethernet port.

An Ethernet switch can be included having at least three ports including at least the first Ethernet port and the second Ethernet port. The Ethernet switch can be configurable to pass data received from the data-network using the second Ethernet port to the medical-analysis station using the first Ethernet port without modification of the data by the at least one processor. An Ethernet switch can be included having at least three ports including at least the first Ethernet port and the second Ethernet port. The Ethernet switch can be configurable to pass processed physiological parameter data received from the medical-analysis station on the first Ethernet port to the data-network using the second Ethernet port without modification of the processed physiological parameter data by the at least one processor.

The medical-analysis station connector can be configured to receive control data in the first format from the medical-analysis station. The plurality of patient-parameter-connectors can be configured to transmit the received control data in the second format to the one or more physiological parameter data generators.

The medical-analysis station can include a bedside monitor, a docking station with a removable monitor, or a therapy device. The one or more physiological parameter data generators can include one or more of: a heart rate sensor, an electrocardiogram sensor, a blood oxygen (SpO2) sensor, a temperature sensor, a respiration sensor, an electroencephalography (EEG) sensor, a blood pressure sensor, a gas sensor, a neuromuscular transmission (NMT) sensor) and a bi-spectral index sensor.

Control data can be received by the Ethernet switch, on the first Ethernet connector, and from the medical-analysis station. A destination physiological parameter data generator can be determined based on the received control data. The received control data can be converted by the data conversion unit to serial data according to a serial protocol. The serial data can be transmitted by one of the plurality of patient-parameter-connectors to the destination physiological parameter data generator.

Processed patient physiological parameter from the medical-analysis station data can be received by at least the Ethernet switch and on the first Ethernet connector. The processed patient physiological parameter data can be transmitted by at least the Ethernet switch and on a second Ethernet connection to a data-network. A destination physiological parameter data generator can be determined based on the received control data. The received control data can be converted from the second format to the first format according to a serial protocol. The converted control data can be transmitted on one of the plurality of patient-parameter-connectors in the first format to the destination physiological parameter data generator.

The first format can be a serial format and the second format is an Ethernet data packet format. control data in the second format can be received on the medical-analysis station port.

Processed patient physiological parameter data can be received on the medical-analysis station port in the second format. The processed patient physiological parameter data can be from the medical-analysis station. The processed patient physiological parameter data can be transmitted on at least the data-network port in the second format to a data-network without modification of the processed patient physiological parameter data.

Data can be received on the data-network port in the second format from a data-network. The received data can be transmitted on the medical-analysis station port in the second format to the medical-analysis station without modification of the data by the at least one processor.

Computer program products are also described that comprise non-transitory computer readable media storing instructions, which when executed by at least one data processors of one or more computing systems, causes at least one data processor to perform operations herein. Similarly, computer systems are also described that may include one or more data processors and a memory coupled to the one or more data processors. The memory may temporarily or permanently store instructions that cause at least one processor to perform one or more of the operations described herein. In addition, methods can be implemented by one or more data processors either within a single computing system or distributed among two or more computing systems.

The subject matter described herein provides many advantages. For example, the current subject matter can connect multiple physiological parameter data generators, a medical analysis station, and a data-network, such as a hospital network, so that communication can occur between different connected components. The medical analysis station and/or the data-network can accumulate physiological data from the physiological parameter data generators for processing or viewing at a single location. The current subject matter can enable physiological parameter data generators to connect and communicate with a data-network, such as a hospital network. Moreover, the current subject matter can enable communication between the data-network and a medical analysis station, as well as enable communication between the medical analysis station and the physiological parameter data generators. The current subject matter can provide connectivity without redesigning existing hardware systems.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a block diagram of an example implementation of a patient parameter communication hub;

FIG. 2 is a system diagram illustrating data flow between and among the patient parameter communication hub, physiological parameter generators, medical-analysis station, and data network; and

FIG. 3 and FIG. 4 are process flow diagrams illustrating a method of exchanging data between physiological parameter data generators and a medical-analysis station.

Like reference symbols in the various drawings indicate like elements.

### DETAILED DESCRIPTION

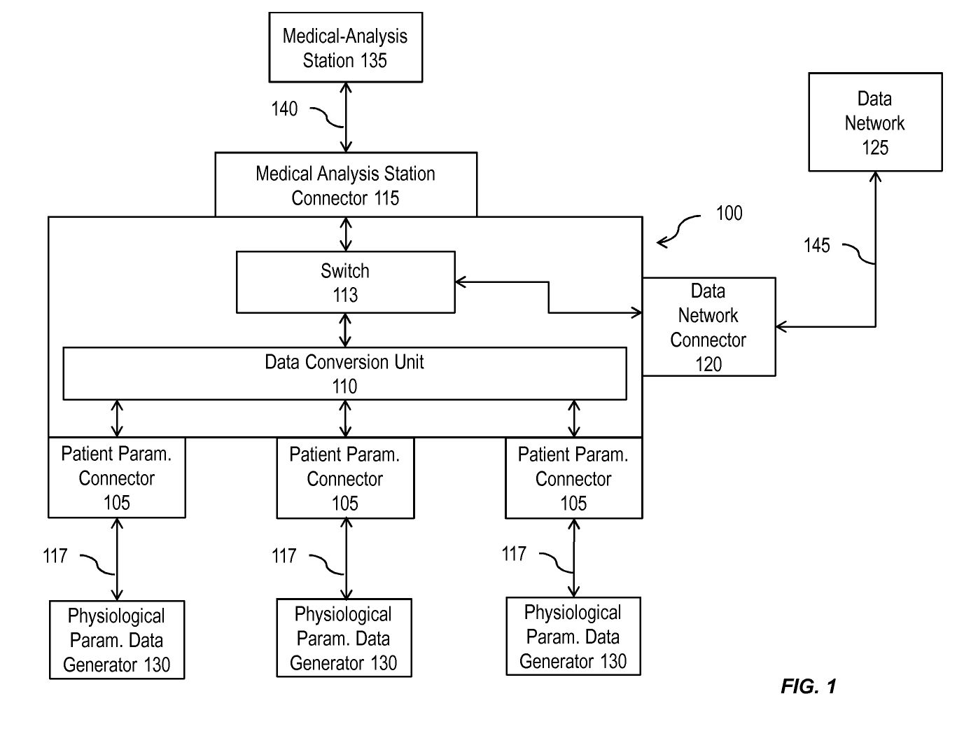

FIG. 1 is a block diagram of an example implementation of a patient parameter communication (PPC) hub 100. The PPC hub 100 can include patient-parameter-connectors 105, data conversion unit 110, switch 113, medical-analysis station connector 115, and data-network connector 120. The PPC hub 100 can be operatively in communication with and can enable data transfer and transmission between physiological parameter data generators 130, medical-analysis station 135, and data network 125. Data such as physiological parameter data, processed physiological parameter data, and control data can be exchanged. Moreover, the PPC hub 100 can convert between data formats. In some implementations, communication between and among physiological parameter data generators 130, medical-analysis station 135, and data network 125 provides flexible patient monitoring configuration and management, as well as physiological parameter data accumulation at locations other than dedicated analysis devices for improved patient monitoring.

The patient-parameter-connectors 105 receive physiological parameter data (e.g., heart rate, blood oxygen, and the like) generated by physiological parameter data generators 130. Data conversion unit 110 converts the format of the received physiological parameter data. Switch 113 can control and/or direct data between the data conversion unit 110, the medical-analysis station connector 115 and the data-network connector 120. Medical-analysis station connector 115 can transmit the received and converted physiological parameter data to a medical-analysis station 135 for processing. The medical analysis-station 135 can process the physiological parameter data to create processed physiological parameter data. The medical-analysis station connector 115 can also receive processed physiological parameter data from the medical-analysis station 135. The data-network connector 120 can transmit and receive data, such as the processed physiological parameter data, over data network 125 to one or more destinations, such as a central viewing and monitoring station, a network database, and/or a remote processing device.

The patient-parameter-connectors 105 receive patient physiological data in a first format from physiological parameter data generators 130 via cables 117. Patient-parameter-connectors 105 can receive data, such as patient physiological data, from the physiological parameter data generators 130 and can transmit data, such as control data, to the physiological parameter data generators 130. In some implementations, the first format is a serial format and the patient-parameter-connectors 105 are serial ports while the cables 117 are serial cables, although other formats are possible.

The data conversion unit 110 can include a microprocessor or other data processor and can convert data between formats. For example, the data conversion unit 110 can convert data received in a first format on the patient-parameter-connectors 105 to a second format and pass the converted data, via switch 113, to the medical-analysis station connector 115 for transmission in the second format. The first format can include serial format and the second format can include Ethernet format. The data conversion unit 110 can convert the physiological parameter data received on the patient-parameter-connectors 105 to data packets according to an Ethernet protocol.

The switch 113 can be an Ethernet switch and can pass data, such as processed physiological parameter data, received from the data network 125 via the data-network connector 120 to medical-analysis station 135 via the medical-analysis station connector 115 without modification or format conversion. The switch 113 can pass data received, such as processed physiological parameter data, received from the medical-analysis station 135 via medical-analysis station connector 115 to the data network 125 via data-network connector 120 without medication or format conversion. The switch 113 can include three ports and, while illustrated separately in FIG. 1, in some implementations the medical-analysis station connector 115 and the data-network connector 120 can form two of the three ports.

The medical-analysis station connector 115 can be operatively in communication with a medical-analysis station 135 via a cable 140. Medical-analysis station connector 115 can transmit data, such as physiological parameter data, to the medical-analysis station 135, and can receive data, such as control data and processed physiological parameter data, from the medical-analysis station 135. In some implementations, the medical-analysis station connector 115 is an Ethernet port and the cable 140 is an Ethernet cable.

The data-network connector 120 can be operatively in communication with a data network 125 via cable 145. Data-network connector 120 can transmit data, such as processed physiological parameter data, to and/or over the data network 125, and receive data, such as further processed physiological parameter data or configuration data, from the data network 125. In some implementations, the data-network connector 120 is an Ethernet port and the cable 145 is an Ethernet cable.

The physiological parameter data generators 130 can include any medical sensor, for example, heart rate sensors, electrocardiogram sensors (ECG), blood oxygen (SPO2) sensors, temperature sensors, respiration sensors, electroencephalography (EEG) sensor, blood pressure sensors, gas sensors, neuromuscular transmission (NMT) sensors, and bispectral index sensors. Other physiological parameter data generators 130 are possible.

The medical-analysis station 135 can include any analysis station, for example, a bedside monitor, a docking station with a removable monitor, and/or a therapy device. Other medical-analysis stations 135 are possible.

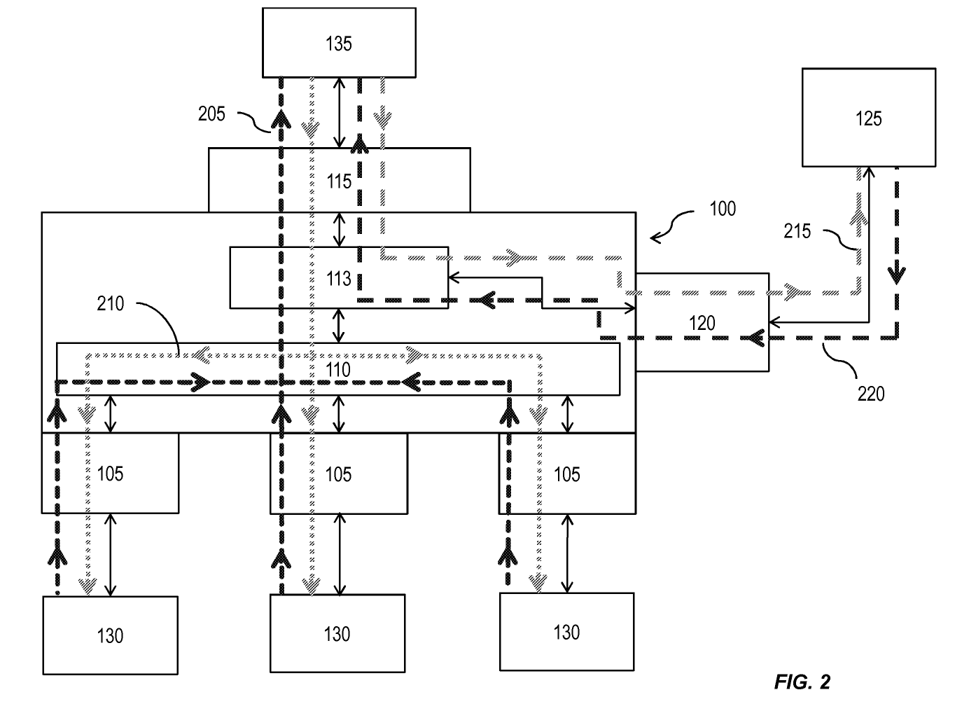

FIG. 2 is a system diagram illustrating data flow between and among the PPC hub 100, physiological parameter data generators 130, medical-analysis station 135, and data network 125.

Data can be transmitted between the physiological parameter data generators 130 and the medical-analysis station 135 via PPC hub 100. For example, data flow line 205 illustrates physiological parameter data sent from each of the physiological parameter data generators 130 to the medical-analysis station 135. Patient-parameter-connectors 105 receive physiological parameter data in a first format, which is converted by the data conversion unit 110 into a second format, which is directed by and passes through the switch 113 to the medical-analysis station connector 115 and transmitted to the medical-analysis station 135.

Data flow line 210 illustrates control data sent from the medical-analysis station 135 to the physiological parameter data generators 130 via the PPC hub 100. Control data can include configuration parameters for the physiological parameter data generators 130, for example, information relating to a patient's age, gender, and device operating settings. The medical-analysis station connector 115 receives control data in the second format, which is passed and/or directed through switch 113 to data conversion unit 110, which converts the control data in the second format to the first format. Patient-parameter-connectors 105 transmit the control data in the first format to one or more of the physiological parameter data generators 130.

Data can be transmitted between the medical-analysis station 135 and the data network 125 via PPC hub 100. For example, data flow line 215 illustrates processed physiological parameter data sent from the medical-analysis station 135 to the data network 125. Medical-analysis station connector 115 receives processed parameter data in the second format, which is passed and/or directed through switch 113 to data-network connector 120, which transmits the unmodified processed parameter data in the second format to the data network 125. Data flow line 220 illustrates the reverse data flow, in which data, such as additionally processed physiological parameter data, is sent from the data network 125 to the medical-analysis station 135. Data-network connector 120 receives further processed parameter data in the second format, which is passed and/or directed through switch 113 to medical-analysis station connector 115, which transmits the unmodified further processed parameter data in the second format to the medical-analysis station 135.

Parameter data from the medical analysis station 135 can be sent to the data network 125 via PPC hub 100 to be consumed by a database processing engine to produce historical data. This historical data can in turn be requested for display on the medical analysis station 135 at later times and can be transmitted to the medical analysis station 135 also via PPC hub 100.

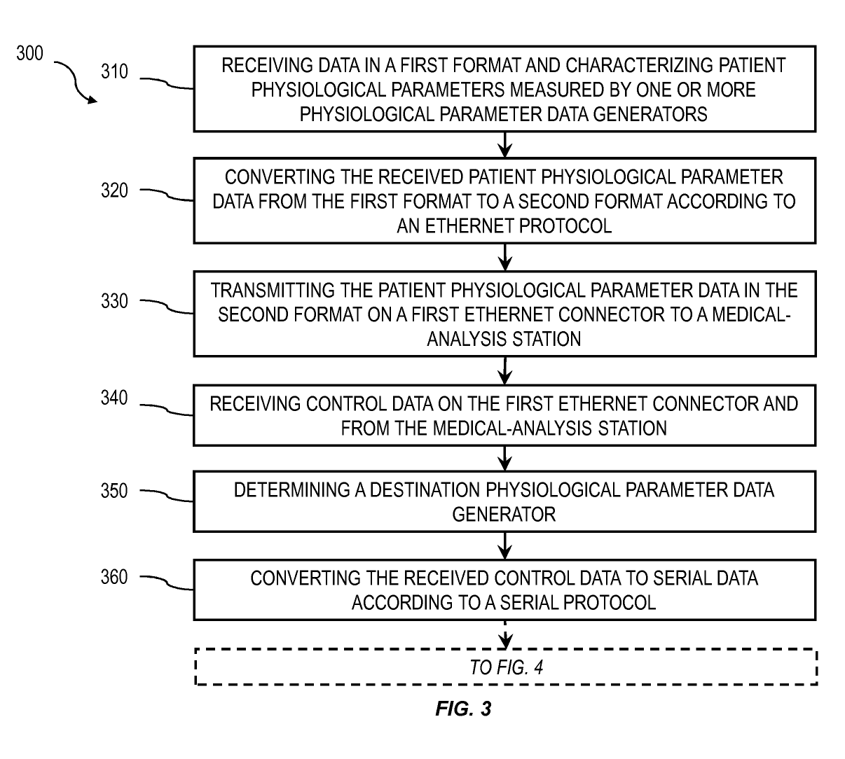

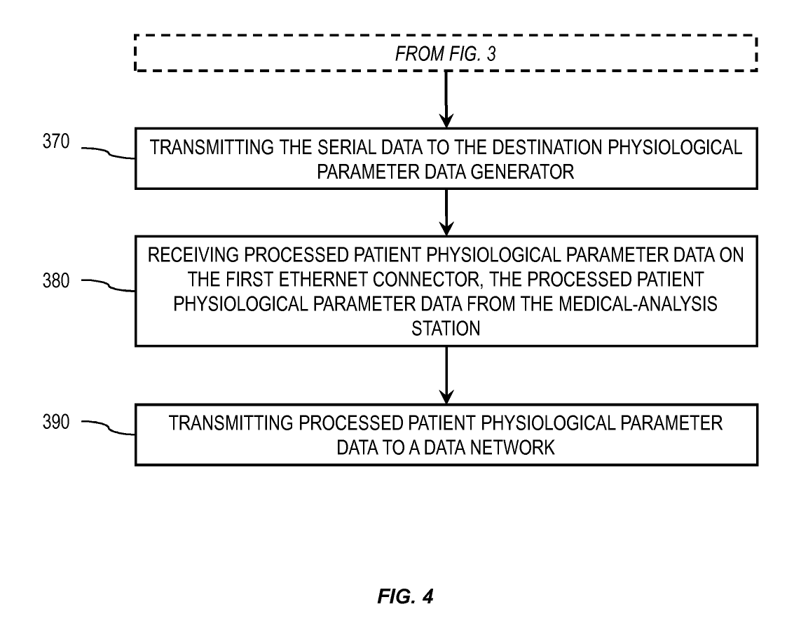

FIG. 3 and FIG. 4 show a process flow diagram illustrating a method 300 of exchanging data between physiological parameter data generators 130 and a medical-analysis station 135. Data is received, at 310 and by one of a plurality of patient-parameter-connectors 105, in a first format and characterizing patient physiological parameters measured by one or more physiological parameter data generators 130. The received patient physiological parameter data is converted, at 320 and by a data conversion unit 110, from the first format to the second format according to an Ethernet protocol. The patient physiological parameter data can be transmitted, at 330 and by at least an Ethernet switch having three or more connectors, in the second format on a first Ethernet connector to a medical-analysis station 135. The first format can include a serial format and the second format can include an Ethernet data packet format.

Control data can be received at 340 by the Ethernet switch on the first Ethernet connector. The control data can be from the medical-analysis station 135. A destination physiological parameter data generator can be determined at 350 and based on the received control data. The received control data can be converted, at 360 and by the data conversion unit 110, to serial data according to a serial protocol. The serial data can be transmitted, at 370 and by one of the patient-parameter-connectors 105, to the destination physiological parameter data generator.

Processed patient physiological parameter data can be received on the first Ethernet connector, at 380 and by at least the Ethernet switch. The processed patient physiological parameter data can be from the medical-analysis station 135. The processed patient physiological parameter data can be transmitted unmodified at 390, by at least the Ethernet switch, and on a second Ethernet connection, to a data network 125.

Various implementations of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user and a keyboard and a pointing device (e.g., a mouse or a trackball) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

Although a few variations have been described in detail above, other modifications are possible. For example, the implementations described above can be directed to various combinations and subcombinations of the disclosed features and/or combinations and subcombinations of several further features disclosed above. In addition, the logic flows depicted in the accompanying figures and described herein do not require the particular order shown, or sequential order, to achieve desirable results. Other embodiments may be within the scope of the following claims.

1. A system comprising:
a plurality of patient-parameter-connectors adapted for receiving physiological parameter data in a first format, the physiological parameter data generated by one or more physiological parameter data generators, wherein the plurality of patient-parameter-connectors comprises a serial port;
a data conversion unit adapted for converting the received physiological parameter data from the first format to a second format, wherein the first format comprises a serial format and the second format comprises an Ethernet format, wherein the data conversion unit comprises at least one processor and executable instructions stored on memory, which, when executed by the at least one processor, causes the at least one processor to convert the physiological parameter data received on one or more of the plurality of patient-parameter-connectors to data packets according to an Ethernet protocol for transmission on a first Ethernet port;
a medical-analysis station connector adapted for transmitting the received physiological parameter data in the second format to a medical-analysis station for processing and for receiving processed physiological parameter data in the second format from the medical-analysis station, wherein the medical-analysis station connector comprises the first Ethernet port;
a data-network connector adapted for transmitting the received processed physiological parameter data in the second format over a data-network to one or more destinations, wherein the data-network connector comprises a second Ethernet port; and
an Ethernet switch having at least three ports comprising at least the first Ethernet port and the second Ethernet port, the Ethernet switch being configurable to pass data received from the data-network using the second Ethernet port to the medical-analysis station using the first Ethernet port without modification of the data by the at least one processor.

2-4. (canceled)

5. The system of claim 1, further comprising an Ethernet switch having at least three ports comprising at least the first Ethernet port and the second Ethernet port, the Ethernet switch being configurable to pass processed physiological parameter data received from the medical-analysis station on the first Ethernet port to the data-network using the second Ethernet port without modification of the processed physiological parameter data by the at least one processor.

6. The system of claim 1, wherein the medical-analysis station connector is configured to receive control data in the first format from the medical-analysis station and the plurality of patient-parameter-connectors is configured to transmit the received control data in the second format to the one or more physiological parameter data generators.

7. The system of claim 1, wherein the medical-analysis station includes a bedside monitor, a docking station with a removable monitor, or a therapy device.

8. The system of claim 1, wherein the one or more physiological parameter data generators include one or more of: a heart rate sensor, an electrocardiogram sensor, a blood oxygen (SpO2) sensor, a temperature sensor, a respiration sensor, an electroencephalography (EEG) sensor, a blood pressure sensor, a gas sensor, a neuromuscular transmission (NMT) sensor) and a bispectral index sensor.

9. A method comprising:
receiving, by one of a plurality of patient-parameter-connectors, data in a first format and characterizing patient physiological parameters measured by one or more physiological parameter data generators;
converting, by a data conversion unit, the received patient physiological parameter data from the first format to a second format according to an Ethernet protocol; and
transmitting, by at least an Ethernet switch having three or more connectors, the patient physiological parameter data in the second format on a first Ethernet connector to a medical-analysis station without modification of the patient physiological parameter data.

10. The method of claim 9, further comprising:
receiving, by the Ethernet switch, control data on the first Ethernet connector and from the medical-analysis station;
determining, based on the received control data and by at least one data processor, a destination physiological parameter data generator;
converting, by the data conversion unit, the received control data to serial data according to a serial protocol; and
transmitting, by one of the plurality of patient-parameter-connectors, the serial data to the destination physiological parameter data generator.

11. The method of claim 9, further comprising:
receiving, by at least the Ethernet switch, processed patient physiological parameter data on the first Ethernet connector, the processed patient physiological parameter data from the medical-analysis station; and
transmitting, by at least the Ethernet switch and on a second Ethernet connection, the processed patient physiological parameter data to a data-network without modification of the patient physiological parameter data.

12. The method of claim 9, wherein the medical-analysis station includes a bedside monitor, a docking station with a removable display, or a therapy device.

13. The method of claim 9, wherein the one or more physiological parameter data generators include one or more of: a heart rate sensor, an electrocardiogram sensor, a blood oxygen (SpO2) sensor, a temperature sensor, a respiration sensor, an electroencephalography (EEG) sensor, a blood pressure sensor, a gas sensor, a neuromuscular transmission (NMT) sensor) and a bi-spectral index sensor.

14. The method of claim 9, wherein the first format is a serial format and the second format is an Ethernet data packet format.

15. A system comprising:
a plurality of serial ports;
an Ethernet switch comprising a medical-analysis station port and a data-network port; and
a data conversion unit coupled to the plurality of serial ports and the Ethernet switch, the data conversion unit comprising at least one processor and executable instructions stored on memory, which, when executed by the at least one processor, causes the at least one processor to perform operations comprising:
receiving, on one of the plurality of patient-parameter-connectors, data in a first format and characterizing patient physiological parameters measured by one or more physiological parameter data generators;
converting, using at least one data processor, the received patient physiological parameter data from the first format to a second format according to an Ethernet protocol; and
transmitting, by the medical-analysis station port, the patient physiological parameter data in the second format to a medical-analysis station without modification of the patient physiological parameter data.

16. The system of claim 15, the operations further comprising:
receiving, on the medical-analysis station port, control data in the second format;
determining, based on the received control data and by at least one data processor, a destination physiological parameter data generator;
converting, using at least one data processor, the received control data from the second format to the first format according to a serial protocol; and
transmitting, on one of the plurality of patient-parameter-connectors, the converted control data in the first format to the destination physiological parameter data generator.

17. The system of claim 15, the operations further comprising:
receiving, on the medical-analysis station port, processed patient physiological parameter data in the second format, the processed patient physiological parameter data from the medical-analysis station; and
transmitting, on at least the data-network port, the processed patient physiological parameter data in the second format to a data-network without modification of the processed patient physiological parameter data by the at least one processor.

18. The system of claim 15, the operations further comprising:
receiving, on the data-network port, data in the second format from a data-network; and
transmitting, on the medical-analysis station port, the received data in the second format to the medical-analysis station without modification of the data by the at least one processor.

19. The system of claim 15, wherein the medical-analysis station includes a bedside monitor, a docking station with a removable display, or a therapy device.

20. The system of claim 15, wherein the one or more physiological parameter data generators include one or more of: a heart rate sensor, an electrocardiogram sensor, a blood oxygen (SpO2) sensor, a temperature sensor, a respiration sensor, an electroencephalography (EEG) sensor, a blood pressure sensor, a gas sensor, a neuromuscular transmission (NMT) sensor) and a bispectral index sensor.

21. The system of claim 15, wherein the first format is a serial format and the second format is an Ethernet data packet format.

22-23. (canceled)

24. The system of claim 1, further comprising the medical-analysis station.

25. The system of claim 1, further comprising the one or more physiological parameter data generators.
